(19) United States
(12) Patent Application Publication
(10) Pub. No.: US 2013/0224304 A1
(43) Pub. Date: Aug. 29, 2013

(54) EFFERVESCENT NUTRITIONAL AND/OR DIETARY SUPPLEMENT COMPOSITION

(71) Applicant: Nordic Naturals, Inc., (US)

(72) Inventor: Joar Opheim, Aptos, CA (US)

(73) Assignee: Nordic Naturals, Inc., Watsonville, CA (US)

(21) Appl. No.: 13/857,812

(22) Filed: Apr. 5, 2013

Related U.S. Application Data
(63) Continuation of application No. 12/392,833, filed on Feb. 25, 2009, now abandoned.

(51) Int. Cl. A23L 1/29; A61K 9/50; A61K 9/46

(52) U.S. Cl. CPC A23L 1/293; A61K 9/0007; A61K 9/5036
USPC 424/493; 424/44; 426/591; 426/2

(57) ABSTRACT

An effervescent composition is provided comprising a dry, free-flowing powder comprising (a) microcapsules comprising a hollow, solid, water soluble outer shell comprising a starch, a sugar or mixtures thereof and an inner core comprising a liquid, water immiscible oil comprising at least one polyunsaturated fatty acid, at least one derivative of a polyunsaturated fatty acid or mixtures thereof, and (b) an effervescing agent.

### CROSS-REFERENCE TO RELATED APPLICATION

This application is a continuation application of U.S. patent application Ser. No. 12/392,833, filed Feb. 25, 2009, which is incorporated herein by reference in its entirety for all purposes.

### BACKGROUND OF THE INVENTION

It has become increasingly important for the prevention and treatment of disease as well as the maintenance of good health for people to supplement the normal intake of food with nutritional and/or dietary supplements. Typically, these substances are required to be taken several times a day in order to fulfill the daily dosage requirements. Many nutritional and/or dietary substances are not well stored by the body requiring frequent dosing. However, the more doses required, the less compliance there is by the patient. People simply will not take the number of pills required to complete the daily dosage requirements. The reasons for failing to take proper dosages include inconvenience, difficulty in swallowing pills, forgetfulness and the like. In addition, the generally poor taste of many nutritional and/or dietary substances adds to the difficulty in completing dosage regimens.

There have been efforts to develop dosage systems which seek to make active ingredients more pleasant and effective for the consumer. In order to provide a pleasant tasting composition and one which provides the desired benefits, it is necessary for the nutritional and/or dietary dosage system meet the following requirements.

The system can provide for both nutritional and/or dietary active ingredients. The formulation must be readily dissolvable in a liquid, particularly water, to provide a pleasant tasting drink. Finally, administration of the composition can be sufficient to provide an optimum delivery of the active ingredients so that the composition is effective until the next dosage which may be as much as approximately 24 hours later.

Efforts have been made to meet the above-stated criteria of a nutritional and/or dietary supplement formulation. For example, U.S. Pat. No. 5,055,306, issued Oct. 8, 1991 to Barry et al., discloses a granular sustained-release formulation of a pharmacologically active substance presented in the form of a tablet, said tablet comprising sufficient granules to provide a predetermined dose or number of doses of the pharmacologically active substance and effervescent or water-dispersible ingredients, each of said granules having a diameter of preferably between 0.5 and 2.5 mm and comprising (a) a core comprising one or more pharmacologically active substances and preferably one or more excipients, and (b) a coating covering substantially the whole surface of the core and comprising 100 parts of a water insoluble but water swellable acrylic polymer and from 20 to 70 parts of a water soluble hydroxylated cellulose derivative, the weight of the coating being from 2 to 25% of the weight of the core.

U.S. Pat. No. 5,178,878, issued Jan. 12, 1993 to Wehling et al., discloses a pharmacological dosage form incorporating microparticles which are susceptible to rupture upon chewing or which are adapted to provide substantially immediate release of the pharmacological ingredient contained in the microparticles. The microparticles are provided as a tablet with an effervescent disintegration agent. When the tablet is taken orally, the effervescent disintegration agent aids in rapid dissolution of the tablet and hence permits release of the microparticles, and swallowing of the microparticles, before the pharmaceutical ingredient is released from the microparticles. The system therefore provides effective taste masking. The pharmaceutical ingredient can be a dietary supplement including cod liver oil or fish oil.

The microparticle may be provided as a microcapsule or as a matrix-type microparticle. Microcapsules typically incorporate a discrete mass of the pharmaceutical ingredient surrounded by a discrete, separately observable coating of the protective material. Conversely, in a matrix-type particle, the pharmaceutical ingredient is dissolved, suspended or otherwise dispersed throughout the protective material. Certain microparticles may include attributes of both microcapsules and matrix-type particle. For example, a microparticle may incorporate a core incorporating a dispersion of the pharmaceutical ingredient in a first protective material and a coating of a second protective material, which may be the same as or different from the first protective material surrounding the core. Alternatively, a microparticle may incorporate a core consisting essentially of the pharmaceutical ingredient and a coating incorporating the protective material, the coating itself having some of the pharmaceutical ingredient dispersed within it.

Prompt release of the microparticles is said to be preferred. However, the protective material utilized in the microparticle desirably should not dissolve instantaneously in water or saliva. That is, the microparticle should resist dissolution and release for a period of time, typically a few seconds or so, sufficient to permit the patient to swallow the released microparticles as the tablet disintegrates. It is said the microparticles made using any of the polymeric protective materials disclosed in the '878 patent will not dissolve instantaneously.

U.S. Pat. No. 5,560,928, issued Oct. 1, 1996 to DeFelice, discloses a composition and method of using the same which provides in as little as a single dose covering a 24 hour period a nutritional and/or dietary supplement which provides administration of water soluble and water insoluble active ingredients for immediate and sustained-release delivery. The dietary supplement can include cod liver oil or fish oil. The active ingredient can be microencapsulated by a process in which the active ingredient is coated with a continuous film of a natural or synthetic polymer. It is said that microencapsulation of a water insoluble substance can function to mask unpleasant tastes but is principally used for slowing down the rate of release of the active ingredient to provide a sustained-release formulation.

U.S. Pat. No. 6,071,539, issued Jun. 6, 2000 to Robinson et al.; U.S. Pat. No. 6,488,961; issued Dec. 3, 2002 to Robinson et al.; and U.S. Pat. No. 6,649,186, issued Nov. 18, 2003 to Robinson et al., each disclose effervescent granules having a controllable rate of effervescence. The granules comprise an acidic agent, an alkaline agent, a hot-melt extrudable binder capable of forming a eutectic mixture with the acidic agent and, optionally, a plasticizer. The effervescent granules are made by a hot-melt extrusion process. Examples of the hot-melt extrudable binders include starch, sugars and invert sugars. The effervescent granules can be formulated in a variety of forms such as a tablet, capsule, suspension, reconstitutable powder base for a carbonated beverage and suppository. The granules can contain a dietary supplement. Examples of dietary supplements include cod liver oil and fish oil.

U.S. Pat. No. 6,811,793, issued Nov. 2, 2004 to Wehling, discloses a tablet that includes stevia, water soluble binder, water soluble lubricant, active agent and effervescent agent. The active agent can be a dietary supplement. Examples of dietary supplements include cod liver oil and fish oils.

U.S. Pat. No. 7,247,324, issued Jul. 24, 2007 to Wehling et al., discloses a method of using guava extract that includes administering alcohol to a mammal, and administering guava extract within no greater than 12 hours of administering the alcohol. The guava extract can be administered as an effervescent powder, powdered drink mix or effervescent granulation. When in the form of a tablet, the effervescent composition preferably includes a binder. Examples of suitable binders include starches.

### SUMMARY OF THE INVENTION

The present invention provides an effervescent composition comprising a dry, free-flowing powder comprising (a) microcapsules comprising a hollow, solid, water soluble outer shell comprising a starch, a sugar or mixtures thereof and an inner core comprising a liquid, water immiscible oil comprising at least one polyunsaturated fatty acid, at least one derivative of a polyunsaturated fatty acid or mixtures thereof, and (b) an effervescing agent.

The present invention further provides a method of administering a liquid, water immiscible oil comprising at least one polyunsaturated fatty acid, at least one derivative of a polyunsaturated fatty acid or mixtures thereof to a subject comprising (1) providing an effervescent composition comprising a dry, free-flowing powder comprising (a) microcapsules comprising a hollow, solid, water soluble outer shell comprising a starch, a sugar or mixtures thereof and an inner core comprising a liquid, water immiscible oil comprising at least one polyunsaturated fatty acid, at least one derivative of a polyunsaturated fatty acid or mixtures thereof, and (b) an effervescing agent, (2) mixing the effervescent composition with an aqueous liquid, and (3) administering the resulting mixture to the subject.

### DETAILED DESCRIPTION OF EMBODIMENTS

### Effervescent Agent

As used herein, “effervescence” means the evolution of bubbles of gas from a liquid as the result of a bubble or gas generating chemical reaction. The bubble or gas generating reaction of the effervescent couple in the effervescent composition of this invention is most often the result of the reaction of an acidic agent and an alkaline agent. The reaction of these two general classes of compounds produces a gas upon contact with water or an aqueous solution.

As used herein, the term “acidic agent” refers to any compound or material that can serve as a proton source and can react with the alkaline agent to form a gas causing a solution containing them to effervesce. The acidic agent can have more than one acid dissociation constant, i.e. more than one acid functional group. The acidic agent can be any organic or inorganic acid in the free acid, acid anhydride and acid salt form. An acidic agent which is in solid state at room temperature and shows pH 4.5 or lower when saturated into water at room temperatures or its acid alkali metal salts (e.g. sodium salt, potassium salt, etc.) can be employed. As the acidic agent for the effervescent compositions of this invention, a compound which is not harmful to animals including man is desirably employed. The acidic agent can be tartaric acid, citric acid, maleic acid, fumeric acid, malic acid, adipic acid, succinic acid, lactic acid, glycolic acid, alpha hydroxy acids, ascorbic acid, amino acids and their alkali metal acid salts. Even in the case of an acid substance such as phosphoric acid or pyrophosphoric acid or other inorganic acids which is liquid or in liquid state at room temperature, when their acid alkali metal salts are solid at room temperature, those acid alkali metal salts can be employed as acidic agents. Among the above-mentioned acidic agents, those having a relatively large acid dissociation constant (103 or more) and a small hygroscopicity (critical humidity at 30° C. is 40% RH or more) are preferably employed. Preferably, the acidic agents dissolves rapidly (if not essentially instantaneously) in water or an aqueous solution.

As used herein, the term “alkaline agent” means an alkaline compound that releases a gas, or causes a solution to effervesce, when exposed to a proton source such as an acidic agent or water. The alkaline agent can be a carbon dioxide gas precursor, an oxygen gas precursor or a chlorine dioxide gas precursor, but is preferably a carbon dioxide gas precursor.

When the alkaline agent is a carbon dioxide precursor, compounds such as carbonate, bicarbonate, sesquicarbonate and hydrogencarbonate salts (in this specification, carbonate and hydrogencarbonate, or bicarbonate, are generically referred to as carbonate) of potassium, lithium, sodium, calcium, ammonium, or L-lysine carbonate, arginine carbonate, sodium glycine carbonate, sodium amino acid carbonate can be used. When the alkaline agent is an oxygen gas precursor, compounds such as anhydrous sodium perborate, effervescent perborate, sodium perborate monohydrate, sodium per-carbonate and sodium dichloroisocyannurate can be used. When the alkaline agent is a chlorine dioxide (ClO₂) precursor, compounds such as sodium hypochlorite and calcium hypochlorite can be used.

Where the effervescent agent includes two mutually reactive components, such as an acidic agent and an alkaline agent, it is preferred, although not necessary, that both components react completely. Therefore, a ratio of components which provides for equal amounts of reaction equivalents is preferred. For example, if the acid used is diprotic, then either twice the amount of a mono-reactive carbonate alkaline agent, or an equal amount of a all-reactive alkaline agent should be used for complete neutralization to be realized. However, in other embodiments of the present invention, the amount of either the acidic agent or the alkaline agent can exceed the amount of the other component. This can be useful to enhance taste and/or performance of an effervescent composition of the present invention containing an overage of either component. By controlling the relative ratio of acidic agent:alkaline agent, the effervescent composition can be used to regulate the pH of their environment.

The ratio of the above-mentioned acidic agent and alkaline agent can also be determined according to the pH required for dissolving an additional ingredient(s) included in an effervescent composition of the present invention. When the solubility of the additional ingredient(s) increases at the acid side, the pH of the solution is lowered by adding the acidic agent in an amount more than equivalent to the alkaline agent. When the solubility of the additional ingredient(s) increases at the basic side, the pH of the solution is raised by adding the alkaline agent in an amount more than equivalent to the acidic agent. In either case, the pH near the acidic agent immediately after the dissolution is low, while the pH near an alkaline agent is high. In a case where the solubility of the additional ingredient(s) does not depend on pH, the ratio of an acidic agent and an alkaline agent can be optionally selected.

The amount of carbon dioxide precursor, i.e. alkaline agent, to be incorporated is proportional to the volume of carbon dioxide gas generated. When it is desired to increase the dissolution rate of an additional ingredient included in an effervescent composition of this invention, it can be advantageous to increase the amount of carbon dioxide precursor accordingly. The amount of effervescent agent is usually selected from the range of from about 1% to about 10%, preferably from about 1.5% to 5% by weight based on the weight of the effervescent composition.

An acidic agent and a carbon dioxide precursor are used respectively in a powdery or granular state, usually 90% or more of them being capable of passing through a 100 mesh (150μ) screen.

### Nutritional, Dietary, Medicinal and Nutraceutical Supplements

The effervescent compositions of the present invention contain a water-immiscible, liquid (at room temperature) nutritional, dietary, medicinal, or nutraceutical supplement (the term “nutraceutical” is a portmanteau of nutrition and pharmaceutical and refers to extracts of foods claimed to have a medicinal effect on human or animal health). Examples of such water-immiscible, liquid supplements include, but are not limited to, marine oils, sea oils, algae based omega-3 fatty acids or derivatives thereof, alpha linolenic acid, Vitamin A, Vitamin D, Vitamin E, hill oil and fish oil. Fish oil is a preferred supplement.

Fish oil is oil derived from the tissues of oily fish. Fish oil is recommended for a healthy diet because it contains the omega-3 polyunsaturated fatty acids, eicosapentaenoic acid (EPA), and docosahexaenoic acid (DHA), precursors to eicosanoids that reduce inflammation throughout the body. Fish do not actually produce omega-3 fatty acids, but instead can accumulate them from consuming microalgae that produce these fatty acids, as is the case with fish like herring, sardines and cod. Cod liver oil is one example of a fish oil.

The oil used in the effervescent compositions of this invention can be diluted prior to microencapsulation with liquid, essential fatty acids. These include, but are not limited to, flax seed oil (omega-3), oleic acid (omega-9), borage oil (omega-6) and primrose oil (omega-6)

### Omega-3 Polyunsaturated Fatty Acids

As used herein, the term “omega-3 polyunsaturated fatty acid(s)” refers to a family of unsaturated fatty carboxylic acids that have in common a carbon-carbon bond in the n-3 position (i.e., the third bond from the methyl end of the molecule). Typically, they contain from about 16 to about 24 carbon atoms and from three to six carbon-carbon double bonds. Omega-3 polyunsaturated acids can be found in nature, and these natural omega-3 polyunsaturated acids frequently have all of their carbon-carbon double bonds in the cis-configuration.

Examples of omega-3 polyunsaturated fatty acids include, but are not limited to, 7,10,13-hexadecatrienoic acid (sometimes abbreviated as 16:3 (n-3)); 9,12,15-octadecatetrienoic acid (α-linolenic acid (ALA), 18:3 (n-3)); 6,9,12,15-octadecatetraenoic acid (stearidonic acid (STD), 18:4 (n-3)); 11,14,17-eicosatrienoic acid (eicosatrienoic acid (ETE), 20:3 (n-3)); 8,11,14,17-eicosatetraenoic acid (eicosatetraenoic acid (ETA), 20:4 (n-3)); 5,8,11,14,17-eicosapentaenoic acid (eicosapentaenoic acid (EPA), (20:5 (n-3)); 7,10,13,16,19-docosapentaenoic acid (docosapentaenoic acid (DPA), 22:5 (n-3)); 4,7,10,13,16,19-docosahexaenoic acid (docosahexaenoic acid (DHA), 22:6 (n-3)); 9,12,15,18,21-tetracosapentaenoic acid (tetracosapentaenoic acid, 24:5 (n-3)); and 6,9,12,15,18,21-tetracosahexaenoic acid (tetracosahexaenoic acid, 24:6 (n-3)).

Eicosapentaenoic acid (EPA) and docosahexaenoic acid (DHA) are found in nature in fish oils, and have been used in a variety of dietary/therapeutic compositions. EPA and DHA are preferred omega-3 polyunsaturated fatty acids in the present invention.

### Omega-3 Polyunsaturated Fatty Acid Derivatives

As used herein, the teem “omega-3 polyunsaturated fatty acid derivative(s)” refers to omega-3 polyunsaturated fatty acids that have been reacted with another compound or otherwise modified so that the omega-3 polyunsaturated fatty acid no longer contains a free carboxylic acid. Examples of omega-3 polyunsaturated fatty acid derivatives include salts, esters (such as ethyl esters) and glycerides of omega-3 polyunsaturated fatty acids.

As used herein, the teen “glyceride” means a glycerol molecule (i.e., OHCH₂CHOHCH₂OH) in which one, two or all three of the hydroxyls have been esterified with a carboxylic acid, e.g., an omega-3 polyunsaturated acid. Thus, “triglyceride” refers to glycerides in which all three hydroxyls on the glycerol have been esterified with (the same or different) carboxylic acids. “Diglyceride” refers to glycerides in which only two of the hydroxyls on the glycerol have been esterified with (the same or different) carboxylic acids. “Monoglyceride” refers to glycerides in which only one hydroxyl on the glycerol has been esterified with a carboxylic acid.

### Making Microcapsules

The microcapsules' hollow, solid, water soluble shell is comprised of sugar and starch. One example of a useful sugar is a glucose sugar such as a glucose syrup. The starch may be sodium octenyl succinate. It is important to note that the shell material is not gelatin. The starch and sugar materials are used in amounts effective to produce a solid, water soluble outer shell and free-flowing microcapsules. The amount of starch and sugar can vary depending upon the particular starch and sugar used, as well as the particular processing conditions employed. Typically, though, the starch and sugar are used in approximately equal amounts. Thus, in one embodiment, the weight ratio of sugar (e.g., glucose syrup) to starch (e.g., sodium octenyl succinate) is about 1.2:1. However, this weight ratio could vary by 15% or more in either direction.

The microcapsules can be made by known microencapsulation techniques. Basically, the sugar/starch material for the outer shell (plus other ingredients if desired) can be dissolved in a suitable solvent such as food grade alcohol (the solvent evaporates from the shell material during the process). The material for the outer shell and the oil (e.g., marine oil diluted with essential fatty acids if desired) are each fed to separate fine spray nozzles which are facing each other inside a suitable vessel. As the two materials contact each other, the shell material surrounds the oil droplets and hardens to form the outer shell. The process is typically conducted at an elevated temperature (but not at a temperature that will damage the oil or shell material), and in an inert atmosphere. The microcapsules can be dried at elevated temperature, sorted by size and collected.

The composition may include about 7.5% to about 50%, for example about 35%, by weight based on the total weight of the composition of oil, e.g., marine oil such as fish oil, within the microcapsules. The composition may also include about 45% to about 90% packaging. Packaging may include, but is not limited to, sugar or gelatin.

Typically, the microcapsules will be about 0.01 to about 0.1 mm in diameter. When added to an aqueous liquid (e.g., water), the outer shell dissolves (preferably essentially instantaneously), thereby releasing the oil.

The composition can also include other ingredients including, e.g., flavor agents, fillers, surfactants (e.g., polysorbate 80 and sodium lauryl sulfate), color agents including, e.g., dyes and pigments, sweeteners, antioxidants and additional ingredients. These other ingredients can be included in the material that forms the hollow, solid, water soluble shell, or may be used separately.

### Flavor Agents

Useful flavor agents include natural and synthetic flavoring sources including, but not limited to, volatile oils, synthetic flavor oils, flavoring aromatics, oils, liquids, oleoresins and extracts derived from plants, leaves, flowers, fruits, stems and combinations thereof. Useful flavor agents include, e.g., citric oils, e.g., lemon, orange, lime and grapefruit, fruit essences including, e.g., apple, pear, peach, banana, grape, berry, strawberry, raspberry, blueberry, blackberry, cherry, plum, pineapple, apricot, and other fruit flavors. Other useful flavor agents include, e.g., aldehydes and esters (e.g., benzaldehyde (cherry, almond)), citral, i.e., alpha-citral (lemon, lime), neral, i.e., beta-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanal (green fruit), 2-dodedenal (citrus, mandarin) and mixtures thereof, chocolate, cocoa, almond, cashew, macadamia nut, coconut, mint, chili pepper, pepper, cinnamon, vanilla, tooty fruity, mango and green tea. Mixtures of two or more flavor agents may also be employed. When a flavor agent is used, the amount employed will depend upon the particular flavor agent used. However, in general, the flavor agent can constitute from about 5% to about 50% by weight of the effervescent composition.

### Color Agents

Useful color agents include, e.g., food, drug and cosmetic (FD&C) colors including, e.g., dyes, lakes, and certain natural and derived colorants. Useful lakes include dyes absorbed on aluminum hydroxide and other suitable carriers. Mixtures of color agents may also be employed. When a color agent is employed, the amount used will depend upon the particular color agent used. However, in general, the color agent can constitute from about 0.5% to about 5% by weight of the effervescent composition.

### Sweetening Agent

Natural and/or artificial sweetening agents can also be added to the effervescent composition. Examples of sweeteners include sugars such as sucrose, glucose, invert sugar, fructose, and mixtures thereof, saccharin and its various salts (e.g., sodium and calcium salt of saccharin), cyclamic acid and its various salts, dipeptide sweeteners (e.g., aspartame), dihydrochalcone, and sugar alcohols including, e.g., sorbitol, sorbitol syrup, mannitol and xylitol, and combinations thereof. Natural sweeteners that can be employed include, but are not limited to, luo han, stevia or mixtures thereof. Luo han sweetener is derived from luo han guo fruit (siraitia grosvenorii) that is mainly found in China. It is about 300 times sweeter by weight than sucrose. Luo han is commercially available from, e.g., Barrington Nutritionals (Harrison, N.Y.). Stevia is derived from a South American herb, Stevia rebaudiana. It can be up to about 300 times sweeter than sucrose. Because luo han and stevia have such a sweet taste, only a small amount need be used in the effervescent composition. When a sweetening agent is employed the amount used will depend upon the particular sweetening agent used. However, in general, the sweetening agent can constitute from about 0.0005% to about 30% by weight of the effervescent composition. When an agent as sweet as stevia or luo han is used, very small amounts of the sweetener (such as about 0.0005% to about 0.1%, about 0.005% to about 0.015%, or about 0.002% to about 0.003% by weight) can be used.

### Additional Ingredients

The effervescent compositions of the present invention can contain additional ingredients. Examples of such additional ingredients include, but are not limited to, vitamins, minerals and/or herbs.

As used herein, the term “vitamin” refers to trace organic substances that are required in the diet. For the purposes of the present invention, the term vitamin(s) include, without limitation, thiamin, riboflavin, nicotinic acid, pantothenic acid, pyridoxine, biotin, folic acid, vitamin B 12, lipoic acid, ascorbic acid, vitamin A, vitamin D, vitamin E and vitamin K. Also included within the term vitamin are the coenzymes thereof. Coenzymes are specific chemical forms of vitamins. Coenzymes include thiamine pyrophosphates (TPP), flavin mononucleotide (FMM), flavin adenine dinucleotive (FAD), Nicotinamide adenine dinucleotide (AND), Nicotinamide adenine dinucleotide phosphate (NADP), Coenzyme A (CoA), Coenzyme Q10 (CoQ10), pyridoxal phosphate, biocytin, tetrahydrofolic acid, coenzyme B12, lipoyllysine, 11-cis-retinal, and 1,25-dihydroxycholecalciferol. The term vitamin(s) also includes choline, camitine, and alpha, beta, and gamma carotenes.

As used herein, the term “mineral” refers to inorganic substances, metals, and the like required in the human diet. Thus, the term “mineral” as used herein includes, without limitation, calcium, iron, zinc, selenium, copper, iodine, magnesium, phosphorus, chromium and the like, and mixtures thereof. Compounds containing these elements are also included in the term “mineral.”

As used herein, the term “herb” refers to organic substances defined as any of various often aromatic plants used especially in medicine or as seasoning. Thus, the term “herb” as used herein includes, but is not limited to, black currant, ginsing, ginko bilboa, cinnamon and the like, as well as mixtures thereof. Compounds containing these elements are also included in the term “herb.”

Other ingredients that can be used include antioxidants, glucosamine and mixtures thereof.

These other ingredients should be used in effective amounts. Depending upon the particular ingredient used, and its desired effect, the effective of these other ingredients can vary considerably.

The effervescent composition can be made by simply mixing the (preferably dry, free-flowing) components. This is preferably done in a very dry, non-oxygen (e.g., nitrogen) environment. Once mixed, the effervescent composition should be packaged quickly.

A typical effervescent composition of the present invention is comprised of about 40% by weight omega-3 polyunsaturated fatty acids or derivatives thereof (such as glycerides, e/g/, triglycerides), about 48% by weight of the sugar/starch shell composition, and about 12% by weight other ingredients. Typically, at least about 10% by weight of the effervescent composition is a mixture of EPA and DHA (or derivatives thereof).

The amount of the effervescent composition of the invention that is effective will vary depending upon the condition being treated, and can be determined by standard clinical techniques. In addition, in vitro or in vivo assays can optionally be employed to help identify optimal dosage ranges. The precise dose to be employed will also depend on the relative amounts of the components of the effervescent compositions of the invention and the seriousness of the condition being treated and should be decided according to the judgment of the practitioner and each subject's circumstances. However, suitable effective dosage amounts for the compositions of the invention typically provide at least about 500 mg of the oil, e.g., marine oil. In one embodiment, the dose provides at least 500 mg of a combination of EPA and DHA or derivatives thereof. Preferably, the combination contains no less than 200 mg each of EPA and DHA or derivatives thereof.

The following examples demonstrate, but do not limit, the present invention.

### EXAMPLES

There are numerous variations on the embodiments of the present invention illustrated in the Examples which are possible in light of the teachings supporting the present invention. It is therefore understood that within the scope of the following claims, the invention may be practiced otherwise than as specifically described or exemplified.

### Example 1

The following table illustrates a composition used to make the microcapsules of the present invention.

| Ingredient | Weight Percent |
|---|---|
| Microcapsule shell material | |
| Glucose syrup | <26% |
| Starch (sodium octenyl succinate) | <22% |
| Mannitol (optional) | <6.5% |
| Sodium ascorbate | <3% |
| Sodium polyphosphate | <1% |
| Mono- and diglyceride of fatty acids | <1% |
| Tricalcium phosphate | <0.3% |
| Lecithin | <0.2% |
| Mixed tocopherol | <0.2% |
| Ascorbyl palmitate | <0.2% |
| Natural flavor | <0.2% |
| Marine oil | |
| Cod liver oil | <40% |

The cod liver oil is microencapsulated in the shell composition using techniques described above. The resulting free-flowing powder is blended with a powdered effervescing agent to produce the effervescent composition of the present invention.

Although specific embodiments of the present invention have been described, it will be understood by those of skill in the art that there are other embodiments that are equivalent to the described embodiments. Accordingly it is to be understood that the invention is not to be limited by the specific illustrated embodiments.

What is claimed is:

1. An effervescent composition comprising a dry, free-flowing powder comprising (a) microcapsules comprising a hollow, solid, water soluble outer shell comprising a starch, a sugar or mixtures thereof and an inner core comprising a liquid, water immiscible oil comprising at least one polyunsaturated fatty acid, at least one derivative of a polyunsaturated fatty acid or mixtures thereof, and (b) an effervescing agent.

2. The effervescent composition of claim 1 wherein the composition is selected from the group consisting of a dietary supplement, nutritional supplement, nutraceutical, medicinal formulation or combinations thereof.

3. The effervescent composition of claim 1 wherein the starch comprises sodium octenyl succinate.

4. The effervescent composition of claim 1 wherein the sugar comprises glucose syrup.

5. The effervescent composition of claim 1 wherein the liquid, water immiscible oil comprises a marine oil.

6. The effervescent composition of claim 5 wherein the marine oil comprises fish oil.

7. The effervescent composition of claim 1 wherein the liquid, water immiscible oil comprises at least one omega-3 polyunsaturated fatty acid, at least one derivative of an omega-3 polyunsaturated fatty acid, or mixtures thereof.

8. The effervescent composition of claim 7 wherein the liquid, water immiscible oil comprises ethyl esters of omega-3 polyunsaturated fatty acids.

9. The effervescent composition of claim 7 wherein the liquid, water immiscible oil comprises glycerides of omega-3 polyunsaturated fatty acids.

10. The effervescent composition of claim 1 wherein the polyunsaturated fatty acid or derivative of a polyunsaturated fatty acid comprises DHA, EPA, derivatives of DHA, derivatives of EPA or mixtures thereof.

11. The effervescent composition of claim 10 wherein the derivative of a polyunsaturated fatty acid is in the form of ethyl esters DHA, ethyl esters of EPA, or mixtures thereof.

12. The effervescent composition of claim 10 wherein the derivative of a polyunsaturated fatty acid is in the form of glycerides of DHA, glycerides of EPA, or mixtures thereof.

13. The effervescent composition of claim 1 wherein the effervescing agent comprises an acidic agent and an alkaline agent.

14. The effervescent composition of claim 1 wherein the effervescent composition comprises about 40% by weight of a liquid, water immiscible oil comprising at least one polyunsaturated fatty acid, at least one derivative of a polyunsaturated fatty acid or mixtures thereof, about 48% by weight of a combination of sugar and starch and about 12% by weight other ingredients.

15. The effervescent composition of claim 14 wherein the effervescent composition comprises about 10% by weight EPA, DHA or derivatives thereof.

16. The effervescent composition of claim 1 further comprising a sweetening agent, flavor agent, color agent or mixture thereof.

17. The effervescent composition of claim 16 wherein the sweetening agent is stevia.

18. A method of administering a liquid, water immiscible oil comprising at least one polyunsaturated fatty acid, at least one derivative of a polyunsaturated fatty acid or mixtures thereof to a subject comprising (1) providing an effervescent composition comprising a dry, free-flowing powder comprising (a) microcapsules comprising a hollow, solid, water soluble outer shell comprising a starch, a sugar or mixtures thereof and an inner core comprising a liquid, water immiscible oil comprising at least one polyunsaturated fatty acid, at least one derivative of a polyunsaturated fatty acid or mixtures thereof, and (b) an effervescing agent, (2) mixing the effervescent composition with an aqueous liquid, and (3) administering the resulting mixture to the subject.

19. The method of claim 18 wherein the effervescent composition is selected from the group consisting of a dietary supplement, nutritional supplement, nutraceutical, medicinal formulation or combinations thereof.

20. The method of claim 18 wherein the starch comprises sodium octenyl succinate.

21. The method of claim 18 wherein the sugar comprises glucose syrup.

22. The method of claim 18 wherein the liquid, water immiscible oil comprises a marine oil.

23. The method of claim 22 wherein the marine oil comprises fish oil.

24. The method of claim 18 wherein the liquid, water immiscible oil comprises at least one omega-3 polyunsaturated fatty acid, at least one derivative of an omega-3 polyunsaturated acid, or mixtures thereof.

25. The method of claim 24 wherein the liquid, water immiscible oil comprises ethyl esters of omega-3 polyunsaturated fatty acids.

26. The method of claim 24 wherein the liquid, water immiscible oil comprises glycerides of omega-3 polyunsaturated fatty acids.

27. The method of claim 18 wherein the polyunsaturated fatty acid or derivative of a polyunsaturated fatty acid comprises DHA, EPA, derivatives of DHA, derivatives of EPA, or mixtures thereof.

28. The method of claim 27 wherein the derivatives of DHA and derivatives of EPA comprise ethyl esters of DHA, ethyl esters of EPA, or mixtures thereof.

29. The method of claim 27 wherein the derivatives of DHA and derivatives of EPA comprise glycerides of DHA, glycerides of EPA, or mixtures thereof.

30. The method of claim 18 wherein the effervescing agent comprises an acidic agent and an alkaline agent.

31. The method of claim 18 wherein the effervescent composition comprises about 40% by weight of a liquid, water immiscible oil comprising at least one polyunsaturated fatty acid, at least one derivative of a polyunsaturated fatty acid or mixtures thereof, about 48% by weight of a combination of sugar and starch and about 12% by weight other ingredients.

32. The method of claim 31 wherein the effervescent composition comprises about 10% by weight EPA, DHA or derivatives thereof.

33. The method of claim 18 wherein the effervescent composition further comprises a sweetening agent, flavor agent, color agent or mixture thereof.

34. The method of claim 33 wherein the sweetening agent is stevia.
